(19) United States
(12) Patent Application Publication
(10) Pub. No.: US 2025/0197341 A1
(43) Pub. Date: Jun. 19, 2025

(54) METHOD FOR INHIBITING CRYSTALLISATION

(71) Applicant: BIOSYNTHIS, SAINT CYR SOUS DOURDAN (FR)

(72) Inventor: Thierry BERNOUD, SAINT CYR SOUS DOURDAN (FR)

(73) Assignee: BIOSYNTHIS, SAINT CYR SOUS DOURDAN (FR)

(21) Appl. No.: 18/683,861

(22) PCT Filed: Aug. 14, 2022
(86) PCT No.: PCT/EP2022/072743

§ 371 Date: Mar. 7, 2025

(30) Foreign Application Priority Data
Aug. 17, 2021 (FR) FR2108737

(51) Int. Cl. C07C 67/62

(52) U.S. Cl. CPC C07C 67/62

(57) ABSTRACT

A method for inhibiting the crystallization of a composition includes at least the following steps: readying a composition whose pour point, as measured according to standard NF T60-105, is less than or equal to 25° C.; adding to the composition a solvent system having at least one alkane of the formula CₙH₂ₙ₊₂, where n≤11; obtaining an ester/alkane mixture whose pour point, as measured according to standard NF T60-105, is less than or equal to 5° C. The invention also relates to an ester/alkane mixture including: a composition having at least one fatty acid ester whose pour point, as measured according to standard NF T60-105, is less than or equal to 25° C.; a solvent system comprising at least one alkane of the formula CₙH₂ₙ₊₂, where n≤11; wherein the pour point thereof, as measured according to standard NF T60-105, is less than or equal to 5° C.

The present invention relates to a process or method for inhibiting the crystallization of a composition comprising at least one fatty acid ester, making it possible to lower the pour point of said composition and enabling it to be in the liquid state at temperatures of less than or equal to 5° C.

By “in the liquid state” we mean a product which begins to flow under its own weight in less than one minute at a temperature of less than or equal to 5° C.

The present invention relates more particularly to a mixture resulting from said method which is characterized in that it comprises at least one fatty acid ester and a solvent system comprising at least one alkane of the formula CₙH₂ₙ₊₂, where n≤11, whose pour point is less than or equal to 5 C.

The present invention also relates to the use of the mixture resulting from said method as a cosmetic formulation having the following properties: clarity, evanescent silicone-type feel, light and non-greasy, colorless and non-odorous.

In the present application, an ester is a chemical compound that is obtained by a reaction between a carboxylic acid or a derivative thereof and an alcohol. During this chemical reaction, a loss of water molecules can be observed during the reaction between the hydroxyl group of the acid and the hydroxyl group of the alcohol.

More precisely, a fatty acid ester is an ester that is obtained from the reaction of a fatty acid of plant origin and an alcohol.

For the purposes of the present invention, the term “solvent system” is understood to mean the combination of solvents comprising at least one alkane of the formula CₙH₂ₙ₊₂, where n≤11. This solvent system can also include alkanes in which n>11, such as dodecane, tridecane, etc.

The solvent system according to the invention is not a suspension or an emulsion, but rather it is monophasic.

In one embodiment, the solvent system according to the invention does not comprise polymers in the solid, undissolved state.

In one embodiment, the solvent system according to the invention does not comprise vinyl acetate polymer in solid and divided form.

In the present application, the term “pour point of a liquid” is understood to mean the temperature below which the liquid loses its fluid properties. In other words, it is the minimum temperature at which the fluid can flow under its own weight. Below this temperature, the fluid will crystallize to form a crystal lattice.

As part of this application, this physical property is measured according to the French standard NF T60-105.

It can also be measured according to international standards ISO 3016 or ASTM D97.

In the present application, “cloud point” is understood to mean the temperature above which a change in clarity appears. This phenomenon reflects the appearance of the first crystals and thus the first solid particles. This cloud point can be measured according to the EN ISO 3015 standard.

The cloud point can also be determined by measuring the turbidity of a liquid using optical methods, such as visual observation with the naked eye, dynamic light scattering, or even spectroscopy.

For the purposes of the present invention, “crystallization” is understood to mean a process consisting of arranging atoms or molecules such that they form a rigid and well-defined crystal lattice in order to minimize their energy state. The smallest entity in the crystal lattice, called a unit cell, is able to accept atoms or molecules to form a macroscopic crystal. During crystallization, atoms and molecules join together at defined angles to form a characteristic crystal shape with smooth surfaces and facets.

Although crystallization can occur in nature, it is also widely used in industry as a separation and purification step in the pharmaceutical and chemical sectors.

However, certain sectors of the chemical industry such as cosmetics use products that crystallize at low temperatures. This phenomenon is called “product freezing.” Freezing leads to difficulties in handling, transferring, and loading the reactors.

Crystallization inhibition processes can be used to avoid this phenomenon.

A “crystallization inhibition process” is understood to mean a process which, by mechanical or chemical action, blocks the crystallization of a product. More precisely, this results in the interruption of crystal formation.

In EP 2736349A1, the process for inhibiting the crystallization of a palm oil triglyceride is carried out through the addition of a lactic acid ester such as lactylated sodium stearyl or lactylated sodium palmitoyl.

In U.S. Pat. No. 9,271,512, the process for inhibiting fatty acid crystals is carried out through the addition of a polyglyceryl ester.

Likewise, in JP 2012097154A, polyglyceryl fatty acid esters prevent the crystallization of vegetable oils, including palm oil.

Application FR3073411A1 discloses a composition for topical application comprising an oil phase, lipophilic polymer, and a volatile hydrocarbon oil. The application neither discloses nor suggests the inhibition of the crystallization of a composition when the temperature is below 5° C., nor the lowering of the pour point. On the contrary, it suggests using a polymer to structure the oil phases in order to achieve a thickening effect. In an example, it discloses in particular an after-shampoo mask comprising a mixture of myristyl stearate and myristyl palmitate and an undecane/tridecane mixture, as well as said polymer. The pour point of the conditioner mask is not measured and is not a cited feature.

Likewise, application ES 2608641 discloses, for example, a cosmetic cream comprising ethylhexyl stearate and a mixture of undecane and tridecane. The pour point of the cosmetic cream is not measured and is not a cited feature.

Application CN 104696707A discloses a pour point depressant for petroleum products for reducing energy consumption and cost for transporting crude oil in a pipeline comprising an inert fluid, a dispersant, and a polymer powder as a pour point depressant. Since crude oil does not include a fatty acid ester, this document cannot be considered to represent the closest prior art and/or a promising starting point.

On the other hand, in certain embodiments of the same application CN 104696707A, the pour point depressor for petroleum products may additionally comprise a stabilizer whose purpose it is to wet or suspend the polymer. The stabilizer may include, particularly from among a list, n-heptane or n-hexane. However, there is no embodiment or incentive to independently add the stabilizer to the crude oil in order to lower the pour point, since this property is provided by the suspended polymer and not by the solvent system.

These solutions of the prior art describe solutions for the inhibition of vegetable oil crystals for applications in the fields of cosmetics, foodstuffs, or biodiesels. However, none of these prior art documents disclose the inhibition of the low-temperature crystallization of fatty acid esters.

Surprisingly, the applicant has succeeded in developing a method for inhibiting crystallization by lowering the pour point of a fatty acid ester through the addition of a solvent system. The mixture obtained by implementing the method satisfies the requirements of the cosmetics industry. Furthermore, the mixture obtained through the implementation of the method has interesting physicochemical properties, being clear, having an evanescent, silicone-type feel, and being light and non-greasy, colorless and non-odorous.

It is therefore understood, within the meaning of the present invention, that the ester/alkane mixture according to the invention has a pour point which is lower than that of the fatty acid ester.

The present invention is a method for inhibiting the crystallization of a composition, at a temperature of less than or equal to 5° C., which is characterized in that it comprises at least the following steps:
a) readying a composition comprising at least one fatty acid ester whose pour point, as measured according to standard NF T60-105, is less than or equal to 25° C.;
b) adding to said composition a solvent system comprising at least one alkane of the formula CₙH₂ₙ₊₂, where n≤11;
c) obtaining an ester/alkane mixture whose pour point, as measured according to standard NF T60-105, is less than or equal to 5° C.

It is therefore the object of the method according to the present invention to inhibit the crystallization of a composition when its temperature is lower than or 5° C.

The present invention also relates to an ester/alkane mixture comprising:
a. a composition comprising at least one fatty acid ester whose pour point, as measured according to standard NF T60-105, is less than or equal to 25° C.;
b. a solvent system comprising at least one alkane of the formula CₙH₂ₙ₊₂, where n≤11;
   characterized in that the pour point of said ester/alkane mixture, as measured according to standard NF T60-105, is less than or equal to 5° C.

The present invention also relates to an ester/alkane mixture consisting of:
a. a composition consisting of at least one fatty acid ester whose pour point, as measured according to standard NF T60-105, is less than or equal to 25° C.;
b. a solvent system comprising at least one alkane of the formula CₙH₂ₙ₊₂, where n≤11;
   characterized in that the pour point of said ester/alkane mixture, as measured according to standard NF T60-105, is less than or equal to 5° C.

The present invention also relates to an ester/alkane mixture consisting of:
a. a composition consisting of at least one fatty acid ester whose pour point, as measured according to standard NF T60-105, is less than or equal to 25° C.;
b. a solvent system consisting of at least one alkane of the formula CₙH₂ₙ₊₂, where n≤11;
   characterized in that the pour point of said ester/alkane mixture, as measured according to standard NF T60-105, is less than or equal to 5° C.

In one embodiment, the method or the ester/alkane mixture according to the invention is characterized in that the composition comprises at least two fatty acid esters.

In one embodiment, the method or the ester/alkane mixture according to the invention is characterized in that the composition comprises at least three fatty acid esters.

In one embodiment, the method or the ester/alkane mixture according to the invention is characterized in that the composition comprises at least four fatty acid esters.

In one embodiment, the method or the ester/alkane mixture according to the invention is characterized in that the at least one fatty acid ester is of plant origin.

In one embodiment, the method or the ester/alkane mixture according to the invention is characterized in that the at least one fatty acid ester is of synthetic origin.

In one embodiment, the composition comprising at least one fatty acid ester consists predominantly of fatty acid esters.

In one embodiment, the composition comprising at least one fatty acid ester contains at least 50% by weight (w/w) of fatty acid esters relative to the total weight of said composition.

In one embodiment, the composition comprising at least one fatty acid ester contains at least 70% by weight (w/w) of fatty acid esters relative to the total weight of said composition.

In one embodiment, the composition comprising at least one fatty acid ester contains at least 90% by weight (w/w) of fatty acid esters relative to the total weight of said composition.

In one embodiment, the composition comprising at least one fatty acid ester contains at least 95% by weight (w/w) of fatty acid esters relative to the total weight of said composition.

The method for inhibiting the crystallization of a composition as defined above comprises the following steps:

A step a), in which a composition is readied whose pour point, as measured according to standard NFT T60-105, is less than or equal to 25° C.

This step is generally carried out at a temperature of less than or equal to 40° C., and more particularly at a temperature of less than or equal to 25° C.

However, according to one embodiment, the method requires a heating step so that the composition is in the liquid state and not in the solid state when the method is carried out.

This heating step is generally carried out at a temperature of greater than or equal to 40° C. and less than or equal to 150° C., more particularly at a temperature of greater than or equal to 50° C. and less than or equal to 100° C.

This heating step is carried out for a period of 1 hour, 3 hours, or even 6 hours.

A step b), in which a solvent system comprising at least one alkane of the formula CₙH₂ₙ₊₂, where n≤11, is added to said composition.

This addition step is generally carried out at a temperature of greater than or equal to 10° C. and less than or equal to 45° C., more particularly at a temperature of greater than or equal to 20° C. and less than or equal to 40° C.

A step c) of obtaining the ester/alkane mixture whose pour point, as measured according to standard NF T60-105, is less than or equal to 5° C.

Note that a homogenization step can be carried out between steps b) and c). The aim of this step is to obtain a homogeneous mixture in order to measure the properties of the ester/alkane mixture obtained under better operating conditions.

This homogenization step is generally carried out at a temperature of greater than or equal to 15° C. and less than or equal to 60° C., more particularly at a temperature of greater than or equal to 20° C. and less than or equal to 40° C.

This homogenization step is carried out for a period of 1 hour, 3 hours, or even 6 hours.

In one embodiment, the method or the ester/alkane mixture according to the invention is characterized in that the at least one fatty acid ester is selected from the group consisting of esters of formula I:

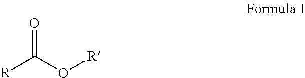

in which
R is selected from the group consisting of linear or branched alkyls comprising m₁ carbon atoms, where m₁ represents an integer selected from the range of 7 to 32;
R′ is selected from the group consisting of linear or branched alkyls comprising m₂ carbon atoms, where m₂ is an integer selected from the range of 2 to 32 carbon atoms; and m₁+m₂>15.

In one embodiment, the method or the ester/alkane mixture according to the invention is characterized in that the at least one fatty acid ester is selected from the group consisting of esters of formula I:

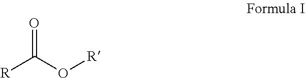

in which
R is selected from the group consisting of linear or branched alkyls comprising m₁ carbon atoms, where m₁ represents an integer selected from the range of 7 to 24;
R′ is selected from the group consisting of linear or branched alkyls comprising m₂ carbon atoms, where m₂ is an integer selected from the range of 2 to 24 carbon atoms; and m₁+m₂>15.

In one embodiment, the method or the ester/alkane mixture according to the invention is characterized in that the at least one fatty acid ester is selected from the group consisting of esters of formula I:

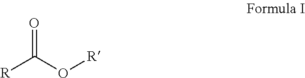

in which
R is selected from the group consisting of linear or branched alkyls and alkenyls comprising m₁ carbon atoms, where m₁ represents an integer selected from the range of 7 to 15;
R′ is selected from the group consisting of linear or branched alkyls comprising m₂ carbon atoms, where m₂ is an integer selected from the range of 2 to 15 carbon atoms; and m₁+m₂>15.

In one embodiment, the method or the ester/alkane mixture according to the invention is characterized in that the at least one fatty acid ester is selected from the group consisting of esters of formula II:

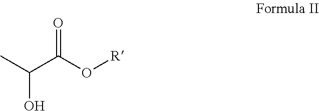

in which
R′ is selected from the group consisting of linear or branched alkyls comprising m₂ carbon atoms, where m₂ is an integer selected from the range of 2 to 32 carbon atoms.

In one embodiment, the method or the ester/alkane mixture according to the invention is characterized in that the at least one fatty acid ester is selected from the group comprising: coco-caprylate/caprate ester (CAS 95912-86-0), ethyl myristate (CAS 124-06-1), ethyl pentadecanoate (41114-00-5), ethyl palmitate (CAS 628-97-7), ethyl heptadecanoate (CAS 14010-23-2), ethyl stearate (CAS 111-61-5), ethyl oleate (CAS 111-62-6), propyl myristate (CAS 14303-70-9), propyl palmitate (CAS 2239-78-3), propyl stearate (CAS 3634-92-2), butyl stearate (CAS 123-95-5), lauryl laurate | (13945-76-1), ethylhexyl palmitate (CAS 29806-73-3), isopropyl palmitate (CAS 142-91-6), 2-octodecyl myristate (CAS 69275-03-2), isopropyl myristate (CAS 110-27-0), 2-ethylhexyl stearate (CAS 22047-49-0), isostearyl lactate (CAS 42131-28-2), and myristyl lactate (CAS 1323-03-1).

For the purposes of the present invention, it will be assumed that the coco-caprylate/caprate ester is produced and not a mixture, since it is identified by CAS number 95912-86-0.

In one embodiment, the method or the ester/alkane mixture according to the invention is characterized in that the at least one fatty acid ester is coco-caprylate/caprate ester.

In one embodiment, the method or the ester/alkane mixture according to the invention is characterized in that the at least one fatty acid ester is myristyl lactate.

In one embodiment, the method according to the invention is characterized in that the at least one fatty acid ester is isopropyl myristate.

In one embodiment, the method or the ester/alkane mixture according to the invention is characterized in that the at least one fatty acid ester is 2-ethylhexyl stearate.

In one embodiment, the method or the ester/alkane mixture according to the invention is characterized in that the at least one fatty acid ester is ethyl stearate and ethyl oleate.

In one embodiment, the method or the ester/alkane mixture according to the invention is characterized in that the composition comprises coco-caprylate/caprate ester.

In one embodiment, the method according to the invention is characterized in that the composition comprises myristyl lactate.

In one embodiment, the method according to the invention is characterized in that the composition comprises isopropyl myristate.

In one embodiment, the method or the ester/alkane mixture according to the invention is characterized in that the composition comprises 2-ethylhexyl stearate.

In one embodiment, the method according to the invention is characterized in that the composition comprises ethyl stearate and ethyl oleate.

In one embodiment, the method or the ester/alkane mixture according to the invention is characterized in that the at least one fatty acid ester has a molecular weight of less than or equal to 600 g/mol.

In one embodiment, the method or the ester/alkane mixture according to the invention is characterized in that the at least one fatty acid ester has a molecular weight of less than or equal to 500 g/mol.

In one embodiment, the method or the ester/alkane mixture according to the invention is characterized in that the at least one fatty acid ester has a molecular weight of less than or equal to 400 g/mol.

For the purposes of the present invention, the pour point of the composition is the pour point of an ester or the pour point of a mixture of esters.

In one embodiment, the method or the ester/alkane mixture according to the invention is characterized in that the composition has a pour point, as measured according to standard NF T60-105, of less than or equal to a temperature of 25° C.

In one embodiment, the method or the ester/alkane mixture according to the invention is characterized in that the composition has a pour point, as measured according to standard NF T60-105, of less than or equal to a temperature of 20° C.

In one embodiment, the method or the ester/alkane mixture according to the invention is characterized in that the composition has a pour point, as measured according to standard NF T60-105, of less than or equal to a temperature of 15° C.

In one embodiment, the method or the ester/alkane mixture according to the invention is characterized in that the composition has a pour point, as measured according to standard NF T60-105, of less than or equal to a temperature of 10° C.

In one embodiment, the method or the ester/alkane mixture according to the invention is characterized in that the composition has a pour point, as measured according to standard NF T60-105, of greater than a temperature of 5° C.

In one embodiment, the method or the ester/alkane mixture according to the invention is characterized in that the composition has a pour point, as measured according to standard NF T60-105, of between 5° C. and 25° C.

In one embodiment, the method or the ester/alkane mixture according to the invention is characterized in that the composition has a pour point, as measured according to standard NF T60-105, of between 5° C. and 20 PC.

In one embodiment, the method or the ester/alkane mixture according to the invention is characterized in that the composition has a pour point, as measured according to standard NF T60-105, of between 5° C. and 15° C.

In one embodiment, the method or the ester/alkane mixture according to the invention is characterized in that the composition has a pour point, as measured according to standard NF T60-105, of between 5° C. and 10° C.

In one embodiment, the method or the ester/alkane mixture according to the invention is characterized in that the composition has a pour point, as measured according to standard NF T60-105, of less than or equal to a temperature of 5° C.

For the purposes of the present invention, the cloud point of the composition, is the cloud point of an ester or the cloud point of a mixture of esters.

In one embodiment, the method or the ester/alkane mixture according to the invention is characterized in that the composition has a cloud point, as measured according to standard EN ISO 3015, of less than or equal to a temperature of 15° C.

In one embodiment, the method or the ester/alkane mixture according to the invention is characterized in that the composition has a cloud point, as measured according to standard EN ISO 3015, of less than or equal to a temperature of 10° C.

In the present application, an “alkane” is a saturated hydrocarbon consisting solely of carbon and hydrogen atoms linked together by single covalent bonds whose general formula is CₙH₂ₙ₊₂; it is referred to as a “linear alkane” if each carbon atom is bonded to no more than two carbon atoms and as a “branched alkane” if certain carbon atoms are bonded to three or even four carbon atoms.

In the present application, a “bioalkane” is a biobased alkane.

In the present application, a compound or an organic composition is considered to be “biobased” if the organic carbon that is present in the compound or composition is of vegetable origin based on a radiocarbon analysis according to one of the following standards: ASTM D6866, EN 16640, or EN 16785-1.

In one embodiment, the method according to the invention is characterized in that the solvent system does not comprise a linear alkane of the formula CₙH₂ₙ₊₂, where n >14.

In one embodiment, the method or the ester/alkane mixture according to the invention is characterized in that the alkane of the formula CₙH₂ₙ₊₂, where n≤11, is selected from the group comprising the linear and/or branched alkanes having no more than 11 carbon atoms, alone or as mixtures.

In one embodiment, the method or the ester/alkane mixture according to the invention is characterized in that the alkane of the formula CₙH₂ₙ₊₂, where n≤11, is selected from the group comprising the linear and/or branched bioalkanes having no more than 11 carbon atoms, alone or as mixtures.

In one embodiment, the method or the ester/alkane mixture according to the invention is characterized in that the alkane of the formula CₙH₂ₙ₊₂, where n≤11, is an alkane whose carbon number is even.

In one embodiment, the method or the ester/alkane mixture according to the invention is characterized in that the alkane of the formula CₙH₂ₙ₊₂, where n≤11, is an alkane whose carbon number is odd.

In one embodiment, the method or the ester/alkane mixture according to the invention is characterized in that the alkane of the formula CₙH₂ₙ₊₂, where n≤11, is selected from the group comprising undecane (CAS 1120-21-4), 2-methyldecane (CAS 6975-98-0), 4-methyldecane (CAS 2847-72-5), 3-methyldecane (13141-34-3), 5-methyldecane (CAS 13151-35-4), 2-6 dimethylnonane (CAS 17302-28-2), 3,7-dimethylnonane (CAS 17302-32-8), 4,5-dimethylnonane (CAS 17302-23-7), 2.3-dimethylnonane (CAS 2884 Jun. 2), 2,4,6-trimethyloctane (CAS 62016-37-9), 2,5,6-trimethyloctane (CAS 62016-14-2), decane (CAS 124-18-5), 2-methylnonane (CAS 871-83-0), 4-methylnonane (CAS 17301-94-9), 3-methylnonane (CAS 5911-04-6), 3-ethyloctane (CAS 5881-17-4), 2,2-dimethyloctane (CAS 15869-87-1), 2,3 dimethyloctane (CAS 7146-60-3), 2,5-dimethyloctane (CAS 15869-89-3), 3,5-dimethyloctane (CAS 15869-93-9), 4-propylheptane (CAS 3178-29-8), 3-ethyl-2-methylheptane (CAS 14676-29-0), 2,2,3-trimethylheptane (CAS 52896-92-1), 2,3,5 trimethylheptane (CAS 20278-85-7), 2,3,6-trimethylheptane (CAS 4032-93-3), 3,3,4-trimethylheptane (CAS 20278-87-9), 2,3,4-trimethylheptane (CAS 52896-95-4), 2,2,4-trimethylheptane (CAS: 14720-74-2) 3,3-diethylhexane (CAS 17302-02-2), 2,2,3,3-tetramethylhexane (CAS 13475-81-5), 3-ethyl-2,2,3-trimethylpentane (CAS 52897-17-3), nonane (111-84-2), 2-methyloctane (3221-61-2), 3-methyloctane (CAS 2216-33-3), 4-methyloctane (CAS 2216-34-4), 2,4-dimethylheptane (CAS 2213-23-2), 2,6-dimethylheptane (CAS 1072 May 5), 2,3-dimethylheptane (CAS 3074-71-3), 2,5-dimethylheptane (CAS 2216-30-0), 2,2-dimethylheptane (CAS 1071-26-7), 2,2,5-trimethylhexane (CAS 352294-9), 2,3,5-trimethylhexane (CAS 1069-53-0), 2,2,4-trimethylhexane (CAS 167476-26-5), 2,3,4-trimethylhexane (CAS 921-47-1), 3-ethylheptane (CAS 15869-80-4), 4-ethylheptane (CAS 2216-32-2), octane (CAS 111-65-9), 2,2,4-trimethylheptane (CAS 592-27-8), 3-methylheptane (CAS 589-81-1), 2-methylheptane (CAS 592-27-8), 2,3,4-trimethylpentane (CAS 565-75-3), 2,4-dimethylhexane (CAS 589-43-5), 2,5-dimethylhexane (CAS 592-13-2), 3,4-dimethylhexane (CAS 583-48-2), 3-ethylhexane (CAS 619-99-8), 4-methylheptane (CAS 589-53-7), and mixtures thereof.

In one embodiment, the method or the ester/alkane mixture according to the invention is characterized in that the alkane of the formula CₙH₂ₙ₊₂, where n≤11, is linear.

In one embodiment, the method or the ester/alkane mixture according to the invention is characterized in that the alkane of the formula CₙH₂ₙ₊₂, where n≤11, is an undecane.

In one embodiment, the method or the ester/alkane mixture according to the invention is characterized in that the alkane of the formula CₙH₂ₙ₊₂, where n≤11, is a decane.

In one embodiment, the method or the ester/alkane mixture according to the invention is characterized in that the alkane of the formula CₙH₂ₙ₊₂, where n≤11, is a nonane.

In one embodiment, the method or the ester/alkane mixture according to the invention is characterized in that the alkane of the formula CₙH₂ₙ₊₂, where n≤11, is an octane.

In one embodiment, the method or the ester/alkane mixture according to the invention is characterized in that the alkane of the formula CₙH₂ₙ₊₂, Where n≤11, is branched.

In one embodiment, the method or the ester/alkane mixture according to the invention is characterized in that the solvent system comprises an alkane of the formula CₙH₂ₙ₊₂, where n≤10, selected from the group comprising decane (CAS 124-18-5), 2-methylnonane (CAS 871-83-0), 4-methylnonane (CAS 17301-94-9), 3-methylnonane (CAS 5911-04-6), 3-ethyloctane (CAS 5881-17-4), 2,2-dimethyloctane (CAS 15869-87-1), 2,3 dimethyloctane (CAS 7146-60-3), 2,5-dimethyloctane (CAS 15869-89-3), 3,5 dimethyloctane (CAS 15869-93-9), 4-propylheptane (CAS 3178-29-8), 3-ethyl-2-methylheptane (CAS 14676-29-0), 2,2,3-trimethylheptane (CAS 52896-92-1), 2,3,5-trimethylheptane (CAS 20278-85-7), 2,3,6-trimethylheptane (CAS 4032-93-3), 3,3,4-trimethylheptane (CAS 20278-87-9), 2,3,4-trimethylheptane (CAS 52896-95-4), 2,2,4-trimethylheptane (CAS 14720-74-2) 3,3-diethylhexane (CAS 17302-02-2), 2,2,3,3-tetramethylhexane (CAS 13475-81-5), 3-ethyl-2,2,3-trimethylpentane (CAS 52897-17-3), nonane (111-84-2), 2-methyloctane (3221-61-2), 3-methyloctane (CAS 2216-33-3), 4-methyloctane (CAS 2216-34-4), 2.4-dimethylheptane (CAS 2213-23-2), 2,6-dimethylheptane (CAS 1072 May 5), 2,3-dimethylheptane (CAS 3074-71-3), 2,5-dimethylheptane (CAS 2216-30-0), 2,2-dimethylheptane (CAS 1071-26-7), 2,2,5-trimethylhexane (CAS 3522-94-9), 2,3,5-trimethylhexane (CAS 1069-53-0), 2,2,4-trimethylhexane (CAS 167476-26-5), 2,3,4-trimethylhexane (CAS 921-47-1), 3-ethylheptane (CAS 15869-80-4), 4-ethylheptane (CAS 2216-32-2), octane (CAS 111-65-9), 2,2,4-trimethylheptane (CAS 592-27-8), 3-methylheptane (CAS 589-81-1), 2-methylheptane (CAS 592-27-8), 2,3,4-trimethylpentane (CAS 565-75-3), 2,4-dimethylhexane (CAS 589-43-5), 2,5-dimethylhexane (CAS 592-13-2), 3,4-dimethylhexane (CAS 583-48-2), 3-ethylhexane (CAS 619-99-8), 4-methylheptane (CAS 589-53-7), and mixtures thereof.

In one embodiment, the method or the ester/alkane mixture according to the invention is characterized in that the solvent system further comprises at least one alkane of the formula CₙH₂₍ₙ₁₎₊₂, where 10≤n1≤14.

The present invention also relates to a ester/alkane mixture consisting of:
a. at least one fatty acid ester whose pour point, as measured according to standard NF T60-105, is less than or equal to 25° C.
b. a solvent system consisting of at least one alkane of the formula CₙH₂ₙ₊₂, where n≤11, and at least one alkane of the formula Cₙ₁H₂₍ₙ₁₎₊₂, where 10≤n1≤14.
   characterized in that the pour point of said ester/alkane mixture, as measured according to standard NF T60-105, is less than or equal to 5° C.

In one embodiment, the method or the ester/alkane mixture according to the invention is characterized in that the alkane of the formula Cₙ₁H₂ₙ₁₊₂, where 10≤n1≤14, is selected from the group comprising the linear and/or branched alkanes having from 10 to 14 carbon atoms, alone or as mixtures.

In one embodiment, the method or the ester/alkane mixture according to the invention is characterized in that the alkane of the formula Cₙ₁H₂ₙ₁₊₂, where 10≤n1≤14, is a linear alkane.

In one embodiment, the method or the ester/alkane mixture according to the invention is characterized in that the alkane of the formula Cₙ₁H₂ₙ₁₊₂, where 10≤n1≤14, is selected from the group comprising the linear and/or branched bioalkanes having from 10 to 14 carbon atoms, alone or as mixtures.

In one embodiment, the method or the ester/alkane mixture according to the invention is characterized in that the alkane of the formula CₙH₂ₙ₁₊₂, where 10≤n1≤14, is a linear bioalkane.

In one embodiment, the method or the ester/alkane mixture according to the invention is characterized in that the alkane of the formula Cₙ₁H₂ₙ₁₊₂, where 10≤n1≤14, is an alkane whose carbon number is even.

In one embodiment, the method or the ester/alkane mixture according to the invention is characterized in that the alkane of the formula Cₙ₁H₂ₙ₁₊₂, where 10≤n1≤14, is an alkane whose carbon number is odd.

In one embodiment, the method or the ester/alkane mixture according to the invention is characterized in that the alkane of the formula CₙH₂₍ₙ₁₎₊₂, where 10≤n1≤14, is decane.

In one embodiment, the method or the ester/alkane mixture according to the invention is characterized in that the alkane of the formula CₙH₂₍ₙ₁₎₊₂, where 10≤n1≤14, is undecane.

In one embodiment, the method or the ester/alkane mixture according to the invention is characterized in that the alkane of the formula CₙH₂₍ₙ₁₎₊₂, where 10≤n1≤14, is dodecane.

In one embodiment, the method or the ester/alkane mixture according to the invention is characterized in that the alkane of the formula Cₙ₁H₂₍ₙ₁₎₊₂, where 10≤n1≤14, is tridecane.

In one embodiment, the method or the ester/alkane mixture according to the invention is characterized in that the alkane of the formula Cₙ₁H₂₍ₙ₁₎₊₂, where 10≤n1≤14, is tetradecane.

In one embodiment, the method or the ester/alkane mixture according to the invention is characterized in that the alkane of the formula Cₙ₁H₂₍ₙ₁₎₊₂, where 10≤n1≤14 is decane and dodecane.

In one embodiment, the method or the ester/alkane mixture according to the invention is characterized in that the alkane of the formula Cₙ₁H₂₍ₙ₁₎₊₂, where 10≤n1≤14 is a branched alkane comprising 10 carbon atoms.

In one embodiment, the method or the ester/alkane mixture according to the invention is characterized in that the branched alkane comprising 10 carbon atoms is selected from the group comprising: 2-methylnonane (CAS 871-83-0), 4-methylnonane (CAS 17301-94-9), 3-methylnonane (CAS 5911-04-6), 3-ethyloctane (CAS 5881-17-4), 2,2-dimethyloctane (CAS 15869-87-1), 2,3-dimethyloctane (CAS 7146-60-3), 2.5-dimethyloctane (CAS 15869-89-3), 3,5 dimethyloctane (CAS 15869-93-9), 4-propylheptane (CAS 3178-29-8), 3-ethyl-2-methylheptane (CAS 14676-29-0), 2,2,3-trimethylheptane (CAS 52896-92-1), 2,3,5-trimethylheptane (CAS 20278-85-7), 2, 3.6-trimethylheptane (CAS 4032-93-3), 3,3,4-trimethylheptane (CAS 20278-87-9), 2,3,4-trimethylheptane (CAS 52896-95-4), 2, 2,4 trimethylheptane (CAS: 14720-74-2), 3,3-diethylhexane (CAS 17302-02-2), 2,2,3,3-tetramethylhexane (CAS 13475-81-5), 3-ethyl-2,2,3-trimethylpentane (CAS 52897-17-3), and mixtures thereof.

In one embodiment, the method or the ester/alkane mixture according to the invention is characterized in that the alkane of the formula Cₙ₁H₂₍ₙ₁₎₊₂, where 10≤n1≤14, is a branched alkane comprising 11 carbon atoms.

In one embodiment, the method according to the invention is characterized in that the branched alkane having 11 carbon atoms is selected from the group comprising: 2-methyldecane (CAS 6975-98-0), 4-methyldecane (CAS 2847-72-5), 3-methyldecane (13141-34-3), 5-methyldecane (CAS 13151-35-4), 2-6-dimethylnonane (CAS 17302-28-2), 3,7-dimethylnonane (CAS 17302-32-8), 3,4-dimethylnonane (CAS 17302-22-6) 4,5 dimethylnonane (CAS 17302-23-7), 4,6 dimethylnonane (CAS 17302-26-0), 3.5 dimethylnonane (CAS 17302-25-9), 2,3-dimethylnonane (CAS 2884 Jun. 2), 2,7 dimethylnonane (CAS 17302-29-3), 2,4,6-trimethyloctane (CAS 62016-37-9), 2,5,6-trimethyloctane (CAS 62016-14-2), 2,3,6-trimethyloctane (CAS 62016-33-5), 2,4,7-trimethyloctane (CAS 62016-38-0), 5-ethylnonane (CAS 17302-12-4), and mixtures thereof.

In one embodiment, the method or the ester/alkane mixture according to the invention is characterized in that the alkane of the formula Cₙ₁H₂₍ₙ₁₎₊₂, where 10≤n1≤14, is a branched alkane comprising 12 carbon atoms.

In one embodiment, the method according to the invention is characterized in that the branched alkane having 12 carbon atoms is selected from the group comprising: 2-methylundecane (CAS 7045-71-8), 3-methylundecane (CAS 1002-43-3), 4-methylundecane (CAS 2980-69-0), 5-methylundecane (CAS 1632-70-8),6-methylundecane (CAS 17302-33-9), 2,4-dimethyldecane (CAS 2801-84-5) 4,4-dimethyldecane (CAS 17312-39-9), 3.5-dimethyldecane (17312-48-0), 2,5-dimethyldecane (CAS 17312-50-4), 2,3-dimethyldecane (17312-44-6), 3,3-dimethyldecane (17302-38-4), 3.7-dimethyldecane (CAS 17312-54-8), 3,4,6-trimethylnonane (CAS 62184-24-1) 3,5,6-trimethylnonane (CAS 62184-26-3), 3,5,7-trimethylnonane (CAS 62184-27-4), 2,5,7-trimethylnonane (CAS 62184-14-9), 2,5,6-trimethylnonane (CAS 62184-13-8), 2,5,7-trimethylnonane (CAS 62184-14-9), 2,5,8-trimethylnonane (CAS 49557-09-7), 3,3,4,5-tetramethyloctane (CAS 62185-21-1), 2,3,4,5-tetramethyloctane (CAS 62199-27-3), 2,2,4,5-tetramethyloctane (CAS 62183-80-6), 2,2,5,7-tetramethyloctane (CAS 62199-19-3), 2,3,4,7-tetramethyloctane (CAS 62199-29-5), 2,4,4,7-tetramethyloctane (CAS 35866-96-7),3-ethyl-4-methylnonane (CAS 62184-45-6), 3-ethyl-4,5-dimethyloctane (CAS 62183-72-6), 2,5-dimethyl-6-ethyloctane (CAS 62183-50-0), and mixtures thereof.

In one embodiment, the method or the ester/alkane mixture according to the invention is characterized in that the alkane of the formula Cₙ₁H₂₍ₙ₁₎₊₂, where 10≤n1≤14, is a branched alkane comprising 13 carbon atoms.

In one embodiment, the method according to the invention is characterized in that the branched alkane having 13 carbon atoms is selected from the group comprising: 2,6-dimethylundecane (CAS 17301-23-4), 2-methyldodecane (CAS 1560-97-0), 4-methyldodecane (CAS 6117-97-1), 4,7-dimethylundecane (CAS 17301-32-5), 3-methyldodecane (CAS 17312-57-1), 3.8-dimethylundecane (CAS 17301-30-3), 2,4,6-trimethyldecane (CAS 62108-27-4), 2,4-dimethylundecane (CAS 17312-80-0), 6-ethyl-2-methyldecane (CAS 62108-21-8), 3.6-dimethylundecane (CAS 17301-28-9), 2,6,7-trimethyldecane (CAS 62108-25-2), 5-methyldodecane (CAS 17453-93-9), 2,5-dimethylundecane (CAS 17301-22-3), 2,3,7-trimethyldecane (CAS 62238-13-5), 3.7-dimethylundecane (CAS 17301-29-0), 2,5,9-trimethyldecane (CAS 62108-22-9), 4,8-dimethylundecane (CAS 17301-33-6), 6-methyldodecane (CAS 6044-71-9), 4.6-dimethylundecane (CAS 17312-82-2), 2,3,5-trimethyldecane (CAS 62238-11-3), 2,5,6-trimethyldecane (CAS 62108-23-0), 2,8-dimethylundecane (CAS 17301-25-6), 2,3-dimethylundecane (CAS 17312-77-5), and 2,6,8-trimethyldecane (CAS 62108-26-3).

In one embodiment, the method or the ester/alkane mixture according to the invention is characterized in that the alkane of the formula Cₙ₁H₂₍ₙ₁₎₊₂, where 10≤n1≤14, is a branched alkane comprising 14 carbon atoms.

In one embodiment, the method according to the invention is characterized in that the branched alkane having 14 carbon atoms is selected from the group comprising: 2-methyltridecane (CAS 1560-96-9), 3-methyltridecane (CAS 6418-41-3), 4-methyltridecane (CAS 26730-12-1), 5-methyltridecane (CAS 25117-31-1), 6-methyltridecane (CAS 13287-21-3), 7-methyltridecane (CAS 26730-14-3) 2,4-dimethyldodecane (CAS 6117-99-3), 2.6-dimethyldecane (CAS 17312-39-9), 3,5-dimethyldodecane (CAS 107770-99-0), 2,5-dimethyldodecane (CAS 61102-34-9), 2,3-dimethyldodecane (CAS 6117-98-2), 2.2-dimethyldodecane (CAS 49598-54-1), 6.7-dimethyldodecane (CAS 20904-59-0), 2,6,10-trimethylundecane (CAS 6864-53-5), 4,6,8-trimethylundecane (CAS 59952-96-4) 2,3,5,8-tetramethyldecane (CAS 192823-15-7), 5,6-diethyldecane (CAS 20904-62-5), 3,8-diethyldecane (CAS 6224-52-8), 4-ethyldodecane (CAS 102636-07-7), 6-propylundecane (CAS 959262-45-4), and mixtures thereof.

In one embodiment, the method or the ester/alkane mixture according to the invention is characterized in that the alkane of the formula Cₙ₁H₂₍ₙ₁₎₊₂, where 10≤n1≤13, is an alkane.

The present invention also relates to an ester/alkane mixture consisting of:
a. at least one fatty acid ester whose pour point, as measured according to standard NF T60-105, is less than or equal to 25° C.
b. a solvent system consisting of at least one alkane of the formula CₙH₂ₙ₊₂, where n≤11, and at least one alkane of the formula Cₙ₁H₂₍ₙ₁₎₊₂, where 10≤n1≤13.
   characterized in that the pour point of said ester/alkane mixture, as measured according to standard NF T60-105, is less than or equal to 5° C.

In one embodiment, the method or the ester/alkane mixture according to the invention is characterized in that the alkane of the formula Cₙ₁H₂₍ₙ₁₎₊₂, where 10≤n1≤13, is selected from the group comprising the linear and/or branched bioalkanes having from 10 to 13 carbon atoms, alone or as mixtures.

In one embodiment, the method or the ester/alkane mixture according to the invention is characterized in that the alkane of the formula CₙH_{2(n1)+2,} where 10≤n1≤13, is decane.

In one embodiment, the method or the ester/alkane mixture according to the invention is characterized in that the alkane of the formula Cₙ₁H₂₍ₙ₁₎₊₂, where 10≤n1≤13, is undecane.

In one embodiment, the method or the ester/alkane mixture according to the invention is characterized in that the alkane of the formula Cₙ₁H₂₍ₙ₁₎₊₂, where 10≤n1≤13, is dodecane.

In one embodiment, the method or the ester/alkane mixture according to the invention is characterized in that the alkane of the formula Cₙ₁H₂₍ₙ₁₎₊₂, where 10≤n1≤13, is tridecane.

In one embodiment, the method or the ester/alkane mixture according to the invention is characterized in that the solvent system further comprises at least one alkane of the formula CₙH₂₍ₙ₂₎₊₂, where 10≤n2≤12.

The present invention also relates to an ester/alkane mixture consisting of:
c. at least one fatty acid ester whose pour point, as measured according to standard NF T60-105, is less than or equal to 25° C.
d. a solvent system consisting of at least one alkane of the formula CₙH₂ₙ₊₂, where n≤11, and at least one alkane of the formula Cₙ₂H₂₍ₙ₂₎₊₂, where 10≤n2≤12.

In one embodiment, the method or the ester/alkane mixture according to the invention is characterized in that the alkane of the formula Cₙ₂H₂₍ₙ₂₎₊₂, where 10≤n2≤12, is selected from the group comprising the linear and/or branched alkanes having from 10 to 12 carbon atoms, alone or as mixtures.

In one embodiment, the method or the ester/alkane mixture according to the invention is characterized in that the alkane of the formula Cₙ₂H₂₍ₙ₂₎₊₂, where 10≤n2≤12, is an alkane with an even carbon number.

In one embodiment, the method or the ester/alkane mixture according to the invention is characterized in that the alkane of the formula Cₙ₂H₂₍ₙ₂₎₊₂, where 10≤n2≤12, is an alkane with an odd carbon number.

In one embodiment, the method or the ester/alkane mixture according to the invention is characterized in that the alkane of the formula Cn₂H₂₍ₙ₂₎₊₂, where 10≤n2≤12, is selected from the group comprising the linear and/or branched bioalkanes having from 10 to 12 carbon atoms, alone or as mixtures.

In one embodiment, the method or the ester/alkane mixture according to the invention is characterized in that the alkane of the formula Cₙ₂H₂₍ₙ₂₎₊₂, where 10≤n2≤12, is decane.

In one embodiment, the method or the ester/alkane mixture according to the invention is characterized in that the alkane of the formula Cₙ₂H₂₍ₙ₂₎₊₂, where 10≤n2≤12, is undecane.

In one embodiment, the method or the ester/alkane mixture according to the invention is characterized in that the alkane of the formula Cₙ₂H₂₍ₙ₂₎₊₂, where 10≤n2≤12, is dodecane.

In one embodiment, the method or the ester/alkane mixture according to the invention is characterized in that the alkane of the formula Cₙ₂H₂₍ₙ₂₎₊₂, where 10≤n2≤12 is decane and dodecane.

In one embodiment, the method or the ester/alkane mixture according to the invention is characterized in that the alkane of the formula Cₙ₂H₂₍ₙ₂₎₊₂, where 10≤n2≤12, is a branched alkane comprising 10 carbon atoms.

In one embodiment, the method or the ester/alkane mixture according to the invention is characterized in that the branched alkane comprising 10 carbon atoms is selected from the group comprising: 2-methylnonane (CAS 871-83-0), 4-methylnonane (CAS 17301-94-9), 3-methylnonane (CAS 5911-04-6), 3-ethyloctane (CAS 5881-17-4), 2,2-dimethyloctane (CAS 15869-87-1), 2,3-dimethyloctane (CAS 7146-60-3), 2,5-dimethyloctane (CAS 15869-89-3), 3,5-dimethyloctane (CAS 15869-93-9), 4-propylheptane (CAS 3178-29-8), 3-ethyl-2-methylheptane (CAS 14676-29-0), 2,2,3-trimethylheptane (CAS 52896-92-1), 2,3,5-trimethylheptane (CAS 20278-85-7), 2,3,6-trimethylheptane (CAS 4032-93-3), 3,3,4-trimethylheptane (CAS 20278-87-9), 2,3,4-trimethylheptane (CAS 52896-95-4), 2,2,4-trimethylheptane (CAS 14720-74-2) 3,3-diethylhexane (CAS 17302-02-2), 2,2,3,3-tetramethylhexane (CAS 13475-81-5), 3-ethyl-2,2,3-trimethylpentane (CAS 52897-17-3), and mixtures thereof.

In one embodiment, the method or the ester/alkane mixture according to the invention is characterized in that the alkane of the formula Cₙ₂H₂₍ₙ₂₎₊₂, where 10≤n2≤12, is a branched alkane comprising 11 carbon atoms.

In one embodiment, the method according to the invention is characterized in that the branched alkane having 11 carbon atoms is selected from the group comprising: 2-methyldecane (CAS 6975-98-0), 4-methyldecane (CAS 2847-72-5), 3-methyldecane (13141-34-3), 5-methyldecane (CAS 13151-35-4), 2-6 dimethylnonane (CAS 17302-28-2), 3,7-dimethylnonane (CAS 17302-32-8), 3,4-dimethylnonane (CAS 17302-22-6) 4,5 dimethylnonane (CAS 17302-23-7), 4,6 dimethylnonane (CAS 17302-26-0), 3,5-dimethylnonane (CAS 17302-25-9), 2,3-dimethylnonane (CAS 2884 Jun. 2), 2,7-dimethylnonane (CAS 17302-29-3), 2,4,6-trimethyloctane (CAS 62016-37-9), 2,5,6-trimethyloctane (CAS 62016-14-2), 2,3,6-trimethyloctane (CAS 62016-33-5), 2,4,7-trimethyloctane (CAS 62016-38-0), 5-ethylnonane (CAS 17302-12-4), and mixtures thereof.

In one embodiment, the method or the ester/alkane mixture according to the invention is characterized in that the alkane of the formula Cₙ₂H₂₍ₙ₂₎₊₂, where 10≤n2≤12, is a branched alkane comprising 12 carbon atoms.

In one embodiment, the method according to the invention is characterized in that the branched alkane having 12 carbon atoms is selected from the group comprising: 2-methylundecane (CAS 7045-71-8), 3-methylundecane (CAS 1002-43-3), 4-methylundecane (CAS 2980-69-0), 5-methylundecane (CAS 1632-70-8),6-methylundecane (CAS 17302-33-9), 2,4-dimethyldecane (CAS 2801-84-5) 4.4-dimethyldecane (CAS 17312-39-9), 3.5-dimethyldecane (17312-48-0), 2,5-dimethyldecane (CAS 17312-50-4), 2,3-dimethyldecane (17312-44-6), 3,3-dimethyldecane (17302-38-4), 3,7-dimethyldecane (CAS 17312-54-8), 3,4,6-trimethylnonane (CAS 62184-24-1) 3,5,6-trimethylnonane (CAS 62184-26-3), 3,5,7-trimethylnonane (CAS 62184-27-4), 2,5,7-trimethylnonane (CAS 62184-14-9), 2,5,6-trimethylnonane (CAS 62184-13-8), 2,5,7-trimethylnonane (CAS 62184-14-9), 2,5,8-trimethylnonane (CAS 49557-09-7), 3,3,4,5-tetramethyloctane (CAS 62185-21-1), 2,3,4,5-tetramethyloctane (CAS 62199-27-3), 2,2,4,5-tetramethyloctane (CAS 62183-80-6), 2,2,5,7-tetramethyloctane (CAS 62199-19-3), 2,3,4,7-tetramethyloctane (CAS 62199-29-5), 2,4,4,7-tetramethyloctane (CAS 35866-96-7), 3-ethyl-4-methylnonane (CAS 62184-45-6), 3-ethyl-4,5-dimethyloctane (CAS 62183-72-6), 2,5-dimethyl-6-ethyloctane (CAS 62183-50-0), and mixtures thereof.

In the present application, a “percentage by weight” is the ratio of the volume of a first compound relative to the total volume of a mixture of compounds or composition, expressed as a percentage.

In one embodiment, the method or the ester/alkane mixture according to the invention is characterized in that the percentage by volume of alcanes of the formula CₙH₂ₙ₊₂, where n≤11, is at least 5% relative to the total volume of the solvent system.

In one embodiment, the method or the ester/alkane mixture according to the invention is characterized in that the percentage by volume of alcanes of the formula CₙH₂ₙ₊₂, where n≤11, is at least 15% relative to the total volume of the solvent system.

In one embodiment, the method or the ester/alkane mixture according to the invention is characterized in that the percentage by volume of alcanes of the formula CₙH₂ₙ₊₂, where n≤11, is between 5 and 70% relative to the total volume of the solvent system.

In one embodiment, the method or the ester/alkane mixture according to the invention is characterized in that the percentage by volume of alcanes of the formula CₙH₂ₙ₊₂, where n≤11, is between 5 and 50% relative to the total volume of the solvent system.

In one embodiment, the method or the ester/alkane mixture according to the invention is characterized in that the percentage by volume of alcanes of the formula CₙH₂ₙ₊₂, where n≤11, is between 5 and 30% relative to the total volume of the solvent system.

In one embodiment, the method or the ester/alkane mixture according to the invention is characterized in that the percentage by volume of branched alkanes having 9 carbon atoms is at least 5% relative to the total volume of the solvent system.

In one embodiment, the method or the ester/alkane mixture according to the invention is characterized in that the percentage by volume of branched alkanes having 9 carbon atoms is at least 15% relative to the total volume of the solvent system.

In one embodiment, the method or the ester/alkane mixture according to the invention is characterized in that the percentage by volume of branched alkanes having 9 carbon atoms is between 1 and 50% relative to the total volume of the solvent system.

In one embodiment, the method or the ester/alkane mixture according to the invention is characterized in that the percentage by volume of branched alkanes having 9 carbon atoms is between 1 and 30% relative to the total volume of the solvent system.

In one embodiment, the method or the ester/alkane mixture according to the invention is characterized in that the percentage by volume of branched alkanes having 9 carbon atoms is at least 5% relative to the total volume of the solvent system.

In one embodiment, the method or the ester/alkane mixture according to the invention is characterized in that the percentage by volume of branched alkanes having 9 carbon atoms is at least 15% relative to the total volume of the solvent system.

In one embodiment, the method or the ester/alkane mixture according to the invention is characterized in that the percentage by volume of branched alkanes having 10 carbon atoms is between 1 and 50% relative to the total volume of the solvent system.

In one embodiment, the method or the ester/alkane mixture according to the invention is characterized in that the percentage by volume of branched alkanes having 10 carbon atoms is between 1 and 30% relative to the total volume of the solvent system.

In one embodiment, the method or the ester/alkane mixture according to the invention is characterized in that the percentage by volume of branched alkanes having 11 carbon atoms is at least 5% relative to the total volume of the solvent system.

In one embodiment, the method or the ester/alkane mixture according to the invention is characterized in that the percentage by volume of branched alkanes having 11 carbon atoms is at least 15% relative to the total volume of the solvent system.

In one embodiment, the method or the ester/alkane mixture according to the invention is characterized in that the percentage by volume of branched alkanes having 11 carbon atoms is between 1 and 50% relative to the total volume of the solvent system.

In one embodiment, the method or the ester/alkane mixture according to the invention is characterized in that the percentage by volume of branched alkanes having 11 carbon atoms is between 1 and 30% relative to the total volume of the solvent system.

In one embodiment, the method or the ester/alkane mixture according to the invention is characterized in that the percentage by volume of nonane is at least 5% relative to the total volume of the solvent system.

In one embodiment, the method or the ester/alkane mixture according to the invention is characterized in that the percentage by volume of nonane is at least 10% relative to the total volume of the solvent system.

In one embodiment, the method or the ester/alkane mixture according to the invention is characterized in that the percentage by volume of nonane is between 5 and 30% relative to the total volume of the solvent system.

In one embodiment, the method or the ester/alkane mixture according to the invention is characterized in that the percentage by volume of nonane is between 5 and 15% relative to the total volume of the solvent system.

In one embodiment, the method or the ester/alkane mixture according to the invention is characterized in that the percentage of decane by volume is at least 5% relative to the total volume of the solvent system.

In one embodiment, the method or the ester/alkane mixture according to the invention is characterized in that the percentage of decane by volume is at least 15% relative to the total volume of the solvent system.

In one embodiment, the method or the ester/alkane mixture according to the invention is characterized in that the percentage of decane by volume is between 5 and 70% relative to the total volume of the solvent system.

In one embodiment, the method or the ester/alkane mixture according to the invention is characterized in that the percentage of decane by volume is between 5 and 50% relative to the total volume of the solvent system.

In one embodiment, the method or the ester/alkane mixture according to the invention is characterized in that the percentage of decane by volume is between 5 and 30% relative to the total volume of the solvent system.

In one embodiment, the method or the ester/alkane mixture according to the invention is characterized in that the percentage of undecane by volume is at least 5% relative to the total volume of the solvent system.

In one embodiment, the method or the ester/alkane mixture according to the invention is characterized in that the percentage of undecane by volume is at least 15% relative to the total volume of the solvent system.

In one embodiment, the method or the ester/alkane mixture according to the invention is characterized in that the percentage of undecane by volume is between 5 and 70% relative to the total volume of the solvent system.

In one embodiment, the method or the ester/alkane mixture according to the invention is characterized in that the percentage of undecane by volume is between 5 and 50% relative to the total volume of the solvent system.

In one embodiment, the method or the ester/alkane mixture according to the invention is characterized in that the percentage of undecane by volume is between 5 and 30% relative to the total volume of the solvent system.

In one embodiment, the method or the ester/alkane mixture according to the invention is characterized in that the percentage of dodecane by volume is at least 40% relative to the total volume of the solvent system.

In one embodiment, the method or the ester/alkane mixture according to the invention is characterized in that the percentage of dodecane by volume is at least 50% relative to the total volume of the solvent system.

In one embodiment, the method or the ester/alkane mixture according to the invention is characterized in that the percentage of dodecane by volume is between 40 and 95% relative to the total volume of the solvent system.

In one embodiment, the method or the ester/alkane mixture according to the invention is characterized in that the percentage of dodecane by volume is between 50 and 85% relative to the total volume of the solvent system.

In one embodiment, the method or the ester/alkane mixture according to the invention is characterized in that the percentage of tridecane by volume is at least 2% relative to the total volume of the solvent system.

In one embodiment, the method or the ester/alkane mixture according to the invention is characterized in that the percentage of tridecane by volume is at least 5% relative to the total volume of the solvent system.

In one embodiment, the method or the ester/alkane mixture according to the invention is characterized in that the percentage of tridecane by volume is between 2 and 30% relative to the total volume of the solvent system.

In one embodiment, the method or the ester/alkane mixture according to the invention is characterized in that the percentage of tridecane by volume is between 5 and 15% relative to the total volume of the solvent system.

In one embodiment, the method or the ester/alkane mixture according to the invention is characterized in that the percentage of tetradecane by volume is at least 2% relative to the total volume of the solvent system.

In one embodiment, the method or the ester/alkane mixture according to the invention is characterized in that the percentage of tetradecane by volume is at least 5% relative to the total volume of the solvent system.

In one embodiment, the method or the ester/alkane mixture according to the invention is characterized in that the percentage of tetradecane by volume is between 2 and 30% relative to the total volume of the solvent system.

In one embodiment, the method or the ester/alkane mixture according to the invention is characterized in that the percentage of tetradecane by volume is between 5 and 15% relative to the total volume of the solvent system.

In one embodiment, the method or the ester/alkane mixture according to the invention is characterized in that the percentage of branched alkanes by volume having 12 carbon atoms is at least 10% relative to the total volume of the solvent system.

In one embodiment, the method or the ester/alkane mixture according to the invention is characterized in that the percentage of branched alkanes by volume having 12 carbon atoms is at least 25% relative to the total volume of the solvent system.

In one embodiment, the method or the ester/alkane mixture according to the invention is characterized in that the percentage of branched alkanes by volume having 12 carbon atoms is between 10 and 50% relative to the total volume of the solvent system.

In one embodiment, the method or the ester/alkane mixture according to the invention is characterized in that the percentage of branched alkanes by volume having 12 carbon atoms is between 10 and 25% relative to the total volume of the solvent system.

In one embodiment, the method or the ester/alkane mixture according to the invention is characterized in that the percentage of branched alkanes by volume having 13 carbon atoms is at least 2% relative to the total volume of the solvent system.

In one embodiment, the method or the ester/alkane mixture according to the invention is characterized in that the percentage of branched alkanes by volume having 13 carbon atoms is at least 5% relative to the total volume of the solvent system.

In one embodiment, the method or the ester/alkane mixture according to the invention is characterized in that the percentage of branched alkanes by volume having 13 carbon atoms is between 2 and 30% relative to the total volume of the solvent system.

In one embodiment, the method or the ester/alkane mixture according to the invention is characterized in that the percentage of branched alkanes by volume having 13 carbon atoms is between 5 and 15% relative to the total volume of the solvent system.

In one embodiment, the method or the ester/alkane mixture according to the invention is characterized in that the percentage of branched alkanes by volume having 14 carbon atoms is at least 2% relative to the total volume of the solvent system.

In one embodiment, the method or the ester/alkane mixture according to the invention is characterized in that the percentage of branched alkanes by volume having 14 carbon atoms is at least 5% relative to the total volume of the solvent system.

In one embodiment, the method or the ester/alkane mixture according to the invention is characterized in that the percentage of branched alkanes by volume having 14 carbon atoms is between 2 and 30% relative to the total volume of the solvent system.

In one embodiment, the method or the ester/alkane mixture according to the invention is characterized in that the percentage of branched alkanes by volume having 14 carbon atoms is between 5 and 15% relative to the total volume of the solvent system.

In the present application, a “percent by weight” is the ratio of the mass of a first compound relative to the total mass of a mixture of compounds or composition, expressed as a percentage.

In one embodiment, the ester/alkane mixture according to the invention or the mixture obtained in step c) of the method according to the invention is characterized in that the ratio Y:

$$Y = \frac{e s t e r ( s ) mass percentage}{alkane ( s ) mass percentage}$$

is at least 9/1.

The percentage of alkane(s) by weight includes the cumulative percentage by weight of alkanes of the formula CₙH₂ₙ₊₂, where n≤11, alkanes of the formula Cₙ₁H₂ₙ₁₊₂, where 10≤n1≤14, alkanes of the formula Cₙ₂H₂₍ₙ₂₎₊₂, where 10≤n2≤12, and/or alkanes of the formula Cₙ₁H₂₍ₙ₁₎₊₂, where 10≤n1≤13.

In one embodiment, the percentage of alkane(s) by weight consists of the cumulative percentage by weight of alkanes of the formula CₙH₂ₙ₊₂, where n≤11, alkanes of the formula Cₙ₁H₂ₙ₁₊₂, where 10≤n1≤14, alkanes of the formula Cₙ₂H₂₍ₙ₂₎₊₂, where 10≤n2≤12, and/or alkanes of the formula Cₙ₁H₂₍ₙ₁₎₊₂, where 10≤n1≤13.

The percentage of ester(s) by weight includes the cumulative percentage by weight of the esters of formula I and formula II.

In one embodiment, the percentage by weight of ester(s) includes the cumulative percentage by weight of coco-caprylate/caprate ester (CAS 95912-86-0), ethyl myristate (CAS 124-06-1), ethyl pentadecanoate (41114-00-5), ethyl palmitate (CAS 628-97-7), ethyl heptadecanoate (CAS 14010-23-2), ethyl stearate (CAS 111-61-5), ethyl oleate (CAS 111-62-6), propyl myristate (CAS 14303-70-9), propyl palmitate (CAS 2239-78-3), propyl stearate (CAS 3634-92-2), butyl stearate (CAS 123-95-5), lauryl laurate | (13945-76-1), ethylhexyl palmitate (CAS 29806-73-3), palmitate d isopropyl (CAS 142-91-6), 2-octodecyl myristate (CAS 69275-03-2), isopropyl myristate (CAS 110-27-0), 2-ethylhexyl stearate (CAS 22047-49-0), isostearyl lactate (CAS 42131-28-2), and/or myristyl lactate (CAS 1323 Mar. 1).

In one embodiment, the percentage by weight of ester(s) consists of the cumulative percentage by weight of the esters of formula I and of formula II.

In one embodiment, the percentage by weight of ester(s) consists of the cumulative percentage by weight of coco-caprylate/caprate ester (CAS 95912-86-0), ethyl myristate (CAS 124-06-1), ethyl pentadecanoate (41114-00-5), ethyl palmitate (CAS 628-97-7), ethyl heptadecanoate (CAS 14010-23-2), ethyl stearate (CAS 111-61-5), ethyl oleate (CAS 111-62-6), propyl myristate (CAS 14303-70-9), propyl palmitate (CAS 2239-78-3), propyl stearate (CAS 3634-92-2), butyl stearate (CAS 123-95-5), lauryl laurate | (13945-76-1), ethylhexyl palmitate (CAS 29806-73-3), isopropyl palmitate (CAS 142-91-6), 2-octodecyl myristate (CAS 69275-03-2), isopropyl myristate (CAS 110-27-0), 2-ethylhexyl stearate (CAS 22047-49-0), isostearyl lactate (CAS 42131-28-2), and myristyl lactate (CAS 1323-03-1).

In one embodiment, the method or the ester/alkane mixture according to the invention is characterized in that the ratio Y is no more than 6/1.

In one embodiment, the method or the ester/alkane mixture according to the invention is characterized in that the ratio Y is no more than 5/1.

In one embodiment, the method or the ester/alkane mixture according to the invention is characterized in that the ratio Y is no more than 4/1.

In one embodiment, the method or the ester/alkane mixture according to the invention is characterized in that the ratio Y is no more than 3/1.

In one embodiment, the method or the ester/alkane mixture according to the invention is characterized in that the ratio Y ranges from 9/1 to 1/1.

In one embodiment, the method or the ester/alkane mixture according to the invention is characterized in that the ratio Y is 9/1.

In one embodiment, the method or the ester/alkane mixture according to the invention is characterized in that the ratio Y is 5/1.

In one embodiment, the method or the ester/alkane mixture according to the invention is characterized in that the ratio Y is 3/1.

In one embodiment, the method or the ester/alkane mixture according to the invention is characterized in that the ratio Y is 2/1.

In one embodiment, the ester/alkane mixture according to the invention or the mixture obtained in step c) of the method according to the invention predominantly comprises fatty acid esters and alkanes of the formula CₙH₂ₙ₊₂, where n≤11, alkanes of the formula Cₙ₁H₂ₙ₁₊₂, where 10≤n1≤14, alkanes of the formula Cₙ₂H₂₍ₙ₂₎₊₂, where 10≤n2≤12, and/or alkanes of the formula Cₙ₁H₂₍ₙ₁₎₊₂, where 10≤n1≤13.

In one embodiment, the ester/alkane mixture according to the invention or the mixture obtained in step c) of the method according to the invention contains at least 50% (w/w) by cumulative weight of fatty acid esters and alkanes of the formula CₙH₂ₙ₊₂, where n≤11, alkanes of the formula Cₙ₁H₂ₙ₁₊₂, where 10≤n1≤14, alkanes of the formula Cₙ₂H₂₍ₙ₂₎₊₂, where 10≤n2≤12, and/or alkanes of the formula Cₙ₃H₂₍ₙ₃₎₊₂, where 10≤n 3≤13, relative to the total weight of the mixture or product.

In one embodiment, the ester/alkane mixture according to the invention or the mixture obtained in step c) of the method according to the invention contains at least 70% (w/w) by cumulative weight of fatty acid esters and alkanes of the formula CₙH₂ₙ₊₂, where n≤11, alkanes of the formula Cₙ₁H₂ₙ₁₊₂, where 10≤n1≤14, alkanes of the formula Cₙ₂H₂₍ₙ₂₎₊₂, where 10≤n2≤12, and/or alkanes of the formula Cₙ₃H₂₍ₙ₃₎₊₂, where 10≤n 3≤13, relative to the total weight of the mixture or product.

In one embodiment, the ester/alkane mixture according to the invention the mixture obtained in step c) of the method according to the invention contains at least 90% (w/w) by cumulative weight of fatty acid esters and alkanes of the formula CₙH₂ₙ₊₂, where n≤11, alkanes of the formula Cₙ₁H₂ₙ₁₊₂, where 10≤n1≤14, alkanes of the formula Cₙ₂H₂₍ₙ₂₎₊₂, where 10≤n2≤12, and/or alkanes of the formula Cₙ₃H₂₍ₙ₃₎₊₂, where 10≤n3≤13, relative to the total weight of the mixture or product.

In one embodiment, the ester/alkane mixture according to the invention or the mixture obtained in step c) of the method according to the invention contains at least 95% (w/w) by cumulative weight of fatty acid esters and alkanes of the formula CₙH₂ₙ₊₂, where n≤11, alkanes of the formula Cₙ₁H₂ₙ₁₊₂, where 10≤n1≤14, alkanes of the formula Cₙ₂H₂₍ₙ₂₎₊₂, where 10≤n2≤12, and/or alkanes of the formula Cₙ₃H₂₍ₙ₃₎₊₂, where 10≤n 3≤13, relative to the total weight of the mixture or product.

In one embodiment, the method according to the invention is characterized in that the mixture obtained in step c) is monophasic.

In one embodiment, the method according to the invention is characterized in that the mixture obtained in step c) has a pour point, as measured according to standard NF T60-105, of less than or equal to a temperature of 5° C.

In one embodiment, the method according to the invention is characterized in that the mixture obtained in step c) has a pour point, as measured according to standard NF T60-105, of less than or equal to a temperature of 0° C.

In one embodiment, the method according to the invention is characterized in that the mixture obtained in step c) has a pour point, as measured according to standard NF T60-105, of less than or equal to a temperature of −5° C.

In one embodiment, the method according to the invention is characterized in that the mixture obtained in step c) has a pour point, as measured according to standard NF T60-105, of less than or equal to a temperature of −10° C.

In one embodiment, the method according to the invention is characterized in that the mixture obtained in step c) has a pour point, as measured according to standard EN ISO 3015, of less than or equal to a temperature of 10° C.

In one embodiment, the method according to the invention is characterized in that the mixture obtained in step c) has a pour point, as measured according to standard EN ISO 3015, of less than or equal to a temperature of 5° C.

In one embodiment, the method according to the invention is characterized in that the mixture obtained in step c) has a pour point, as measured according to standard EN ISO 3015, of less than or equal to a temperature of 0° C.

In one embodiment, the ester/alkane mixture according to the invention is characterized in that it has a pour point, as measured according to standard NF T60-105, of less than or equal to a temperature of 5° C.

In one embodiment, the ester/alkane mixture according to the invention is characterized in that it has a pour point, as measured according to standard NF T60-105, of less than or equal to a temperature of 0° C.

In one embodiment, the ester/alkane mixture according to the invention is characterized in that it has a pour point, as measured according to standard NF T60-105, of less than or equal to a temperature of −5° C.

In one embodiment, the ester/alkane mixture according to the invention is characterized in that it has a pour point, as measured according to standard NF T60-105, of less than or equal to a temperature of −10° C.

In one embodiment, the ester/alkane mixture according to the invention is characterized in that it has a cloud point, as measured according to standard EN ISO 3015, of less than or equal to a temperature of 10° C.

In one embodiment, the ester/alkane mixture according to the invention is characterized in that it has a cloud point, as measured according to standard EN ISO 3015, of less than or equal to a temperature of 5° C.

In one embodiment, the ester/alkane mixture according to the invention is characterized in that it has a cloud point, as measured according to standard EN ISO 3015, of less than or equal to a temperature of 0° C.

In one embodiment, the method according to the invention is characterized in that it comprises a homogenization step between step b), in which the solvent system is added to the composition, and step c), in which the mixture is obtained.

In one embodiment, the method according to the invention is characterized in that step b), in which the solvent system is added to the composition, is carried out at a temperature of at least 10° C.

In one embodiment, the method according to the invention is characterized in that step b), in which the solvent system is added to the composition, is carried out at a temperature of at least 20° C.

In one embodiment, the method according to the invention is characterized in that step b), in which the solvent system is added to the composition, is carried out at a temperature of no more than 40° C.

Another object according to the present invention relates to the use of a solvent system comprising at least one alkane of the formula CₙH₂ₙ₊₂, where n≤11, to inhibit the crystallization of a composition at a temperature of less than or equal to 5° C.

Another object according to the present invention relates to the use of a solvent system comprising at least one alkane of the formula CₙH₂ₙ₊₂, where n≤11, to lower the pour point of a composition to a temperature of less than or equal to 5° C.

Another object according to the present invention relates to the use of a solvent system comprising at least one alkane of the formula CₙH₂ₙ₊₂, where n≤11, to inhibit, at a temperature of less than or equal to 5° C., the crystallization of a composition comprising at least one fatty acid ester whose pour point, as measured according to standard NF T60-105, is less than or equal to 25° C.

In one embodiment of any one of the uses according to the present invention, the alkane of the formula CₙH₂ₙ₊₂, where n≤11, is as defined above.

In one embodiment of any one of the uses according to the present invention, the alkane of the formula CₙH₂ₙ₊₂, where n≤11, is selected from the group comprising the linear and/or branched bioalkanes having no more than 11 carbon atoms, alone or as mixtures.

In one embodiment of any one of the uses according to the present invention, the alkane of the formula CₙH₂ₙ₊₂, where n≤11, is a decane.

In one embodiment of any one of the uses according to the present invention, the solvent system is as defined above.

In one embodiment of any one of the uses according to the present invention, the solvent system further comprises at least one alkane of the formula CₙH₂₍ₙ₁₎₊₂, where 10≤n1≤14.

In one embodiment of any one of the uses according to the present invention, the alkane of the formula CₙH₂ₙ₁₊₂, where 10≤n1≤14, is selected from the group comprising the linear and/or branched bioalkanes having no more than 10 to 14 carbon atoms, alone or as mixtures.

In one embodiment of any one of the uses according to the present invention, the alkane of the formula Cₙ₁H₂₍ₙ₁₎₊₂, where 10≤n1≤14, is a dodecane.

In one embodiment of any one of the uses according to the present invention, the alkane of the formula Cₙ₁H₂₍ₙ₁₎₊₂, where 10≤n1≤13, is an alkane.

In one embodiment of any one of the uses according to the present invention, the solvent system further comprises at least one alkane of the formula CₙH₂₍ₙ₂₎₊₂, where 10≤n2≤12.

In one embodiment of any one of the uses according to the present invention, the alkane of the formula CₙH₂₍ₙ₂₎₊₂, where 10≤n2≤12, is selected from the group comprising the linear and/or branched bioalkanes having from 10 to 12 carbon atoms, alone or as mixtures.

In one embodiment of any one of the uses according to the present invention, the volume percentage of alkanes of the formula CₙH₂ₙ₊₂, where n≤11, of the solvent system is at least 5% relative to the total volume of the solvent system.

In one embodiment of any one of the uses according to the present invention, the fatty acid ester is as defined above.

In one embodiment of any one of the uses according to the present invention, the at least one fatty acid ester is selected from the group consisting of the esters of formula I

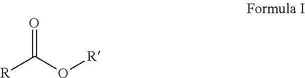

in which
R is selected from the group consisting of linear or branched alkyls comprising m₁ carbon atoms, where m₁ represents an integer selected from the range of 7 to 32;
R′ is selected from the group consisting of linear or branched alkyls comprising m₂ carbon atoms, where m₂ is an integer selected from the range of 2 to 32 carbon atoms; and m₁+m₂>15.

In one embodiment of any one of the uses according to the present invention, the at least one fatty acid ester is selected from the group consisting of the esters of formula II:

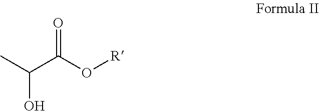

in which
R′ is selected from the group consisting of linear or branched alkyls comprising m₂ carbon atoms, where m₂ is an integer selected from the range of 2 to 32 carbon atoms.

In one embodiment of any one of the uses according to the present invention, the at least fatty acid ester is selected from the group comprising: coco-caprylate/caprate ester (CAS 95912-86-0), ethyl myristate (CAS 124-06-1), ethyl pentadecanoate (41114-00-5), ethyl palmitate (CAS 628-97-7), ethyl heptadecanoate (CAS 14010-23-2), ethyl stearate (CAS 111-61-5), ethyl oleate (CAS 111-62-6), propyl myristate (CAS 14303-70-9), palmitate propyl (CAS 2239-78-3), propyl stearate (CAS 3634-92-2), butyl stearate (CAS 123-95-5), lauryl laurate | (13945-76-1), ethylhexyl palmitate (CAS 29806-73-3), isopropyl palmitate (CAS 142-91-6), 2-octodecyl myristate (CAS 69275-03-2), isopropyl myristate (CAS 110-27-0), 2-ethylhexyl stearate (CAS 22047-49-0), isostearyl lactate (CAS 42131-28-2), and myristyl lactate (CAS 1323 Mar. 1).

In the context of the present application, the method is intended to be used in the field of the cosmetics industry.

However, it is entirely possible for a similar or identical method to be employed in the field of the biofuels industry or even the food industry.

The uses of the ester/alkane mixture obtained by means of the method according to the invention are uses that are aimed at incorporating said mixture into cosmetic formulations.

For example, the ester/alkane mixture could be used in oil formulations, also commonly referred to as oil or lipophilic phases. The ester/alkane mixture can also be used in the lipophilic phases of emulsions.

The cosmetic formulations are intended for facial, body, or hair care.

More specifically, formulations for hair include formulations such as hair creams, hair care products, styling products, coloring products, or even straightening products.

Likewise, formulations for the face include formulations such as makeup formulations, makeup remover formulations, facial care (masks, creams, scrub products, depigmentation products), moisturizing formulations, UV protection, anti-aging formulations, and anti-wrinkle formulations.

Makeup formulations are more particularly formulations which include: mascaras, foundations, eye shadow, eyeliner, concealer formulations, and lipsticks.

Facial formulations may also include formulations for treating or slowing skin damage due to burns or sunburn.

Facial formulations may also include formulations for treating skin redness.

Finally, body formulations include anti-UV, anti-aging, anti-wrinkle, moisturizing, depigmenting, and pro-pigmenting formulations or nail polishes.

### EXAMPLES

The examples which illustrate the method of the present invention below are in no way limiting.

In the examples that follow, the coco-caprylate/caprate ester has a pour point of 15° C. as measured according to standard NF T60-105.

Likewise, the shea ethyl oleate/stearate used in the following examples has a pour point of 2° C. as measured according to standard NF T60-105.

### COMPARATIVE EXAMPLES

### Example 1: Comparative Example in which the Described Method According to the Invention is Carried Out in the Presence of Dodecane Alone and a Coco-Caprylate/Caprate Ester

75 g of a coco-caprylate/caprate ester composition marketed under the name NEODERM LC by BIOSYNTHIS are readied.

A solvent system—i.e., 25 g of pure dodecane—is added to the composition.

A mixture comprising 25 g of vegetable dodecane and 75 g of a coco-caprylate/caprate ester is thus obtained.

The cloud point is 8° C. as measured according to EN ISO 3015.

The pour point is 6° C. as measured according to standard NF T60-105.

The method carried out in the presence of BIOSYNTHIS dodecane alone does not enable the crystallization of the coco-caprylate/caprate ester to be inhibited at a temperature of less than or equal to 5° C.

### Example 2: Comparative Example in which the Described Method According to the Invention is Carried Out in the Presence of Farnesane (Trimethyldodecane) and a Coco-Caprylate/Caprate Ester

70 g of a coco-caprylate/caprate ester composition marketed under the name NEODERM LC by BIOSYNTHIS are readied.

A solvent system—particularly 30 g of pure farnesane (trimethyldodecane) marketed under the name NEOSSANCE HEMISQUALANE by AMYRIS—is added to the composition.

A mixture comprising 30 g of farnesane (trimethyldodecane) and 70 g of a coco-caprylate/caprate ester is thus obtained.

The cloud point is 8° C. as measured according to EN ISO 3015.

The pour point is 6° C. as measured according to standard NF T60-105.

The method carried out in the presence of farnesane (trimethyldodecane) alone does not enable the crystallization of the coco-caprylate/caprate ester to be inhibited at a temperature of less than or equal to 5° C.

### Inventive Examples

### Example 3: Method According to the Invention in the Presence of Dodecane, Decane, and a Coco-Caprylate/Caprate Ester

70 g of a coco-caprylate/caprate ester composition marketed under the name NEODERM LC by BIOSYNTHIS are readied.

A solvent system—particularly 30 g of a dodecane/decane mixture (24 g of dodecane and 6 g of decane) marketed under the name VEGELIGHT SILK by BIOSYNTHIS—is added to the composition.

A mixture comprising 6 g of decane, 24 g of dodecane, and 70 g of a coco-caprylate/caprate ester is thus obtained.

The cloud point is 6° C. as measured according to EN ISO 3015.

The pour point is 5° C. as measured according to standard NF T60-105.

The mixture obtained according to the method of the invention is in the liquid state at a temperature of 5° C.

Therefore, the method carried out in the presence of a mixture of decane and dodecane enables the crystallization of the coco-caprylate/caprate ester to be inhibited at a temperature of 5° C.

### Example 4: Method According to the Invention in the Presence of Undecane, Tridecane, and a Coco-Caprylate/Caprate Ester

80 g of a coco-caprylate/caprate ester composition marketed under the name NEODERM LC by BIOSYNTHIS are readied.

A solvent system—particularly 20 g of a mixture of undecane/tridecane (14 g of undecane and 6 g of tridecane) marketed under the name CETIOL ULTIMATE by BASF—is added to the composition.

A mixture comprising 14 g of undecane and 6 g of tridecane and 80 g of a coco-caprylate/caprate ester is thus obtained.

The cloud point is 6° C. as measured according to EN ISO 3015.

The pour point is −18° C. as measured according to standard NF T60-105.

The mixture obtained according to the method of the invention is in the liquid state at a temperature of 5° C.

Therefore, the method carried out in the presence of a mixture of undecane and tridecane enables the crystallization of the coco-caprylate/caprate ester to be inhibited at a temperature of less than 5° C.

### Example 5: Described Method According to the Invention in the Presence of Dodecane, Decane, and a Shea Oil Ester (Ethyl Oleate/Stearate)

A composition of 80 g of an oleate/ethyl stearate ester (55 g ethyl oleate, 25 g ethyl stearate) originating from shea oil produced by BIOSYNTHIS is readied.

A solvent system—particularly 20 g of a dodecane/decane mixture (16 g of dodecane and 4 g of decane) marketed under the name VEGELIGHT SILK by BIOSYNTHIS—is added to the composition.

A mixture comprising 4 g of decane, 16 g of dodecane, and 80 g of an ethyl oleate/stearate of shea oil is thus obtained.

The pour point is −12° C. as measured according to standard NF T60-105.

The mixture obtained according to the method of the invention is in the liquid state at a temperature of −10° C.

Therefore, the method carried out in the presence of a mixture of decane and dodecane enables the crystallization of ethyl oleate/stearate originating from shea oil to be inhibited at a temperature of −10° C.

1. A method for inhibiting the crystallization of a composition at a temperature of less than or equal to 5° C., wherein it comprises at least the following steps:
a) readying a composition comprising at least one fatty acid ester whose pour point, as measured according to standard NF T60-105, is less than or equal to 25° C.;
b) adding to that composition a solvent system comprising at least one alkane of the formula CₙH₂ₙ₊₂, where n≤11;
c) obtaining an ester/alkane mixture whose pour point, as measured according to standard NF T60-105, is less than or equal to 5° C.

2. The method according to claim 1, wherein the composition has a pour point, as measured according to standard NF T60-105, of less than or equal to a temperature of 15° C.

3. The method according to claim 1, wherein the at least one fatty acid ester is selected from the group consisting of the esters of formula I 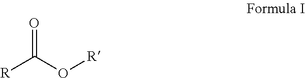
in which
R is selected from the group consisting of linear or branched alkyls comprising m₁ carbon atoms, where m₁ represents an integer selected from the range of 7 to 32;
R′ is selected from the group consisting of linear or branched alkyls comprising m₂ carbon atoms, where m₂ is an integer selected from the range of 2 to 32 carbon atoms; and m₁+m₂>15.

4. The method according to claim 1, wherein the at least one fatty acid ester is selected from the group consisting of the esters of formula II: 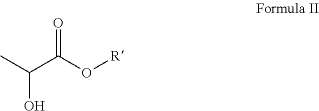
in which
R′ is selected from the group consisting of linear or branched alkyls comprising m₂ carbon atoms, and where m₂ is an integer selected from the range of 2 to 32 carbon atoms.

5. The method according to claim 1, wherein the at least one fatty acid ester is selected from the group comprising: coco-caprylate/caprate ester (CAS 95912-86-0), myristate ethyl (CAS 124-06-1), ethyl pentadecanoate (41114-00-5), ethyl palmitate (CAS 628-97-7), ethyl heptadecanoate (CAS 14010-23-2), ethyl stearate (CAS 111-61-5), ethyl oleate (CAS 111-62-6), propyl myristate (CAS 14303-70-9), propyl palmitate (CAS 2239-78-3), propyl stearate (CAS 3634-92-2), butyl stearate (CAS 123-95-5), lauryl laurate (13945-76-1), palmitate of ethylhexyl (CAS 29806-73-3), isopropyl palmitate (CAS 142-91-6), 2-octodecyl myristate (CAS 69275-03-2), isopropyl myristate (CAS 110-27-0), 2-ethylhexyl stearate (CAS 22047-49-0), isostearyl lactate (CAS 42131-28-2), myristyl lactate (CAS 1323-03-1).

6. The method according to claim 1, wherein the alkane of the formula CₙH₂ₙ₊₂, where n≤11, is selected from the group comprising the linear and/or branched bioalkanes having no more than 11 carbon atoms, alone or as mixtures.

7. The method according to claim 1, wherein the alkane of the formula CₙH₂ₙ₊₂, where n≤11, is a decane.

8. The method according to claim 1, wherein the solvent system further comprises at least one alkane of the formula Cₙ₁H₂₍ₙ₁₎₊₂, where 10≤n1≤14.

9. The method according to claim 8, wherein the alkane of the formula Cₙ₁H₂ₙ₁₊₂, where 10≤n1≤14, is selected from the group comprising linear and/or branched bioalkanes comprising from 10 to 14 carbon atoms, alone or as mixtures.

10. The method according to claim 8, wherein the alkane of the formula Cₙ₁H₂₍ₙ₁₎₊₂, where 10≤n1≤14, is dodecane.

11. The method according to claim 1, wherein the alkane of the formula Cₙ₁H₂₍ₙ₁₎₊₂, where 10≤n1≤13, is an alkane.

12. The method according to claim 1, wherein the solvent system further comprises at least one alkane of the formula Cₙ₂H₂₍ₙ₂₎₊₂, where 10≤n2≤12.

13. The method according to claim 12, wherein the alkane of the formula Cₙ₂H₂₍ₙ₂₎₊₂, where 10≤n2≤12, is selected from the group comprising linear and/or branched bioalkanes comprising from 10 to 12 carbon atoms, alone or as mixtures.

14. The method according to claim 1, wherein the volume percentage of alkanes of the formula CₙH₂ₙ₊₂, where n≤11, of the solvent system is at least 5% relative to the total volume of the solvent system.

15. The method according to claim 1, wherein, in the mixture obtained in step c), the ratio Y
$$Y = \frac{e s t e r ( s ) mass percentage}{alkane ( s ) mass percentage}$$
is no more than 9/1.

16. The method according to claim 1, wherein the mixture obtained in step c) is monophasic.

17. The method according to claim 1, wherein the mixture obtained in step c) has a cloud point, as measured according to standard EN ISO 3015, of less than or equal to a temperature of 10° C.

18. An ester/alkane mixture, comprising:
a) a composition comprising at least one fatty acid ester whose pour point, as measured according to standard NF T60-105, is less than or equal to 25° C.;
b) a solvent system comprising at least one alkane of the formula CₙH₂ₙ₊₂, where n≤11;
wherein the pour point thereof, as measured according to standard NF T60-105, is less than or equal to 5° C.

19. The ester/alkane mixture according to claim 18, wherein the pour point thereof, as measured according to standard NF T60-105, is less than or equal to a temperature of 15° C.

20. The ester/alkane mixture according to claim 18, wherein the at least one fatty acid ester is selected from the group consisting of the esters of formula I 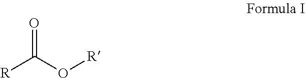
in which
R is selected from the group consisting of linear or branched alkyls comprising m₁ carbon atoms, where m₁ represents an integer selected from the range of 7 to 32;
R′ is selected from the group consisting of linear or branched alkyls comprising m₂ carbon atoms, where m₂ is an integer selected from the range of 2 to 32 carbon atoms; and m₁+m₂>15.

21. The ester/alkane mixture according to claim 18, wherein the at least one fatty acid ester is selected from the group consisting of the esters of formula II: 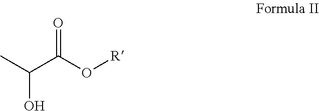
in which
R′ is selected from the group consisting of linear or branched alkyls comprising m₂ carbon atoms, where m₂ is an integer selected from the range of 2 to 32 carbon atoms.

22. The ester/alkane mixture according to claim 18, wherein the at least one fatty acid ester is selected from the group comprising: coco-caprylate/caprate ester (CAS 95912-86-0), ethyl myristate (CAS 124-06-1), ethyl pentadecanoate (41114-00-5), ethyl palmitate (CAS 628-97-7), ethyl heptadecanoate (CAS 14010-23-2), ethyl stearate (CAS 111-61-5), ethyl oleate (CAS 111-62-6), propyl myristate (CAS 14303-70-9), propyl palmitate (CAS 2239-78-3), propyl stearate (CAS 3634-92-2), butyl stearate (CAS 123-95-5), lauryl laurate 1 (13945-76-1), ethylhexyl palmitate (CAS 29806-73-3), isopropyl palmitate (CAS 142-91-6), 2-octodecyl myristate (CAS 69275-03-2), isopropyl myristate (CAS 110-27-0), 2-ethylhexyl stearate (CAS 22047-49-0), isostearyl lactate (CAS 42131-28-2), myristyl lactate (CAS 1323-03-1).

23. The ester/alkane mixture according to claim 18, wherein the alkane of the formula CₙH₂ₙ₊₂, where n≤11, is selected from the group comprising the linear and/or branched bioalkanes having no more than 11 carbon atoms, alone or as mixtures.

24. The ester/alkane mixture according to claim 18, wherein the alkane of the formula CₙH₂ₙ₊₂, where n≤11, is a decane.

25. The ester/alkane mixture according to claim 18, wherein the solvent system further comprises at least one alkane of the formula Cₙ₁H₂₍ₙ₁₎₊₂, where 10≤n1≤14.

26. The ester/alkane mixture according to claim 25, wherein the alkane of the formula Cₙ₁H₂ₙ₁₊₂, where 10≤n1≤14 is selected from the group comprising the linear and/or branched bioalkanes having from 10 to 14 carbon atoms, alone or as mixtures.

27. The ester/alkane mixture according to claim 25, wherein the alkane of the formula Cₙ₁H₂₍ₙ₁₎₊₂, where 10≤n1≤14, is dodecane.

28. The ester/alkane mixture according to claim 25, wherein the alkane of the formula Cₙ₁H₂₍ₙ₁₎₊₂, where 10≤n1≤13, is an alkane.

29. The ester/alkane mixture according to claim 18, wherein the solvent system further comprises at least one alkane of the formula Cₙ₂H₂₍ₙ₂₎₊₂, where 10≤n2≤12.

30. The ester/alkane mixture according to claim 29, wherein the alkane of the formula Cₙ₂H₂₍ₙ₂₎₊₂, where 10≤n2≤12, is selected from the group comprising the linear and/or branched bioalkanes having from 10 to 12 carbon atoms, alone or as mixtures.

31. The ester/alkane mixture according to claim 18, wherein the volume percentage of alkanes of the formula CₙH₂ₙ₊₂, where n≤11, of the solvent system is at least least 5% relative to the total volume of the solvent system.

32. The ester/alkane mixture according to claim 18, wherein the ratio Y
$$Y = \frac{e s t e r ( s ) mass percentage}{alkane ( s ) mass percentage}$$
is no more than 9/1.

33. The ester/alkane mixture according to claim 18, wherein it is monophasic.

34. The ester/alkane mixture according to claim 18, wherein the cloud point thereof, as measured according to standard EN ISO 3015, is less than or equal to a temperature of 10° C.

35. A method comprising
inhibiting, at a temperature of less than or equal to 5° C., crystallization of a composition comprising at least one fatty acid ester whose pour point, as measured according to standard NF T60-105. is less than or equal to 25° C. with a solvent system comprising at least one alkane of the formula CₙH₂ₙ₊₂, where n≤11.

36. (canceled)

37. (canceled)

38. (canceled)
